(19) United States
(12) Patent Application Publication
(10) Pub. No.: US 2021/0253986 A1
(43) Pub. Date: Aug. 19, 2021

(54) METHODS FOR PRODUCING MONOCOTYLEDON WINES AND DERIVATIVE PRODUCTS

(71) Applicant: Onyemaechi Ahanotu, Oakland, CA (US)

(72) Inventor: Onyemaechi Ahanotu, Oakland, CA (US)

(73) Assignee: Onyemaechi Ahanotu, Oakland, CA (US)

(21) Appl. No.: 17/118,617

(22) Filed: Dec. 11, 2020

Related U.S. Application Data
(60) Provisional application No. 62/948,201, filed on Dec. 14, 2019.

(51) Int. Cl. C12G 3/02; A23L 2/38; A23L 2/54; C12N 1/00

(52) U.S. Cl. CPC C12G 3/02; A23L 2/382; A23V 2002/00; C12N 1/00; A23L 2/54

(57) ABSTRACT

Method for producing tropical plant sap based wines (e.g. palm wine, pulque, guarapo) and secondary products derived from these fermented beverages (palm vinegar and distilled spirits). The method comprising of partly or fully supplemented or reconstituted fermentation media to compositions similar to that of naturally occurring sap to be used for the fermentation. Media may be formulated to impart desired beverage characteristics, but primary flavor and beverage properties are derived from products of microbial fermentation (primary or secondary). Fermentation proceeds through any combination of spontaneous, domesticated and/or engineered inoculum. Design of inoculated culture composition to produce desired beverage characteristics.

### RELATED APPLICATIONS

Provisional Patent Application #62/948,201; METHODS OF PRODUCING MONOCOTYLEDONS WINES AND DERIVATIVE PRODUCTS

### BACKGROUND OF THE INVENTION:

Palm wine, pulque and guarapo are all examples of traditional wines found in tropical regions throughout the world, in practice these are based on spontaneous fermentation (uncontrolled inoculation through insect vectors and environmental transport) of sap from select plants in the Glade Monocotyledons (e.g. palms, sugar canes, agave). Fermentation occurs in a mixed culture of wild-type yeast and bacterial microorganisms which are not controlled, other than selection of region, season and type of plant. Plant saps (sap from palm trees in the case of palm wine) are typically collected from the target plant and allowed to incubate/ferment for a short time (3-48 hrs) to convert plant sap sugars into alcohol and other compounds. The uncontrolled micro-organism composition causes, rapid spoilage and highly variable flavor/beverage qualities, both batch-to-batch and over a time span of 24-48 hrs. These beverages spoil or turn to vinegar quickly, sometimes this vinegar is desirable. Prior to complete vinegar conversion, wines may be distilled to make distinctive spirits.

Related wines have been produced for 1000's of years and knowledge of practices tends to be trade specific folk knowledge. Types of plants may be selected due to the flavor profile that they promote. This can be considered a sort of empirical selection of plants to collect sap from.

Some natural materials, such as certain barks, may be added to either promote or suppress growth of unwanted micro-organism. This can be considered using chemical preservatives to reduce spoilage, but while doing so there is a change in beverage properties. Addition of these barks often change appearance and flavor of the resulting products. Tannins are typically the species contributing to increased shelf life. Emerging state of the art includes using thermal pasteurization process (which leads to off-flavors) of young wine that has yet to produce alcohol or acidic bi-products. All aforementioned beverages, as they are now, are not produced to be sparkling.

### SUMMARY OF INVENTION

Fermentation media that is, in large, comprised of reconstituted components, with similar sugar and organic acid concentrations to that of naturally occurring target Monocotyledon sap. This media is formulated to provide a controllable, reproducible and desired chemical composition. Said media is fermented through a tailored inoculum that ferment sugars to alcohols, and produce the primary qualities of the final beverage or food item. This product may undergo further processing such as bottle conditioning or concentrating.

### SUMMARY OF DRAWINGS

FIG. 1—Process flow chart for producing wine, vinegar and/or spirits from reconstituted Monocotyledons sap.

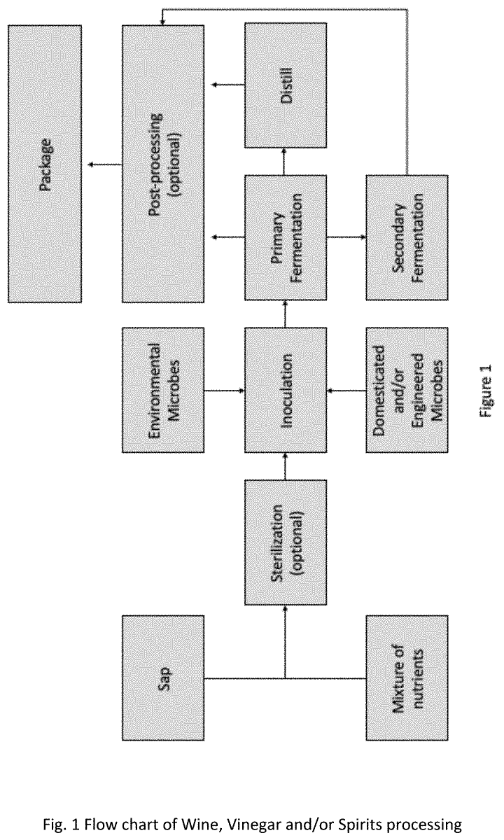

### DETAILED DESCRIPTION OF INVENTION

The present relates to a method for using reconstituted media and tailored inoculum in fermentation to produce food items. In particular to produce fermented material with variable food qualities including flavor, aroma, appearance, chemical composition, shelf-life, and other qualities through directed or controlled fermentation.

Partial or full reconstitution/supplementation of naturally occurring Monocotyledon plant saps, used in producing wine, vinegars, distilled spirits and other food stuffs. Reconstituted or supplemented fermentation media is made up of no more than 75% naturally occurring sap. Reconstituted media may be composed of water, sugars, organic acids, salts, micronutrients, and preservatives. This media is designed to provide nutrients to target micro-organisms of interest, responsible for primary flavor/aroma production, alcohol content, appearance, chemical composition, carbonation and other beverage properties. Media may discourage off-target/spoilage microorganism growth. Media may be used to facilitate primary and/or secondary fermentation.

Media may be sterilized prior to inoculation to reduce contamination. With the partly or fully reconstituted fermentation media, desired micro-organism(s) are added, or inoculated. Fermentation of inoculated media proceeds through controlled or uncontrolled processing. Micro-organisms may be a combination of spontaneous fermentations, domesticated and/or engineered strains.

Target wine, may be bottled with or without addition of preservatives to produce a still or sparkling wine, through a secondary fermentation. Vinegar may be produced from aforementioned wine through bacterial secondary fermentation or the wine may be additionally processed to produce a distilled spirit.

What i claim as my invention is:

1. Beverage, food item or distilled spirit consisting of:
a. a product of fermentation of sugars to produce alcohols, acids and other organic compounds
b. fermentation media containing similar sugar and organic acids compositions to that of the naturally occurring target Monocotyledon sap
c. fermentation media that is either partly or wholly composed of reconstituted components ranging from 25-100% by weight or volume basis
d. the result of fermentation by claim 2.

2. Microbiological fermentation inoculum consisting of:
a. micro-organisms that produce the primary beverage properties of claim 1 ie. flavor, appearance, aroma, chemical composition
b. single organism or mixed species culture used in place of spontaneous inoculation.

3. Carbonated beverage which comprises a bottle conditioning of beverage or food item of claim 1.
